(19) United States
(12) Patent Application Publication
(10) Pub. No.: US 2026/0264063 A1
(43) Pub. Date: Sep. 10, 2026

(54) METAL OXIDE SUPPORTED ATOMIC QUANTUM CLUSTERS (AQCs) CATALYSTS AS OXYGEN CARRIERS FOR CHEMICAL LOOPING PROCESSES

(71) Applicants: NANOGAP SUB-NM-POWDER, S.A., Ames, A Coruna (ES); UNIVERSIDADE DE SANTIAGO DE COMPOSTELA, Santiago de Compostela, A Coruna (ES); TECHNISCHE UNIVERSITAT BERLIN, Berlin (DE); LANCASTER UNIVERSITY, Lancaster (GB)

(72) Inventors: David BUCETA FERNANDEZ, Ames, A Corufia (ES); Anh Dung NGUYEN, Berlin (DE); Qingqing WU, Lancaster (GB); Shahana HUSEYINOVA, Berlin (DE); Reinhard Wilhelm SCHOMACKER, Berlin (DE); Colin John LAMBERT, Lancaster (GB); Manuel Arturo LOPEZ QUINTELA, Santiago de Compostela-A Coruna (ES)

(21) Appl. No.: 18/993,540

(22) PCT Filed: Jul. 11, 2023
(86) PCT No.: PCT/EP2023/069208

§ 371 Date: Jan. 11, 2025

(30) Foreign Application Priority Data
Jul. 12, 2022 (EP) 22382662.9
Apr. 26, 2023 (EP) 23382386.3

(51) Int. Cl. B01J 23/83; B01J 23/63; B01J 23/889; B01J 35/45; B01J 37/02; B01J 37/08; B01J 37/14; B01J 37/16; B01J 37/34; C01B 3/40; C07C 1/12

(52) U.S. Cl. CPC B01J 23/83; B01J 23/63; B01J 23/8892; B01J 35/45; B01J 37/0201; B01J 37/0236; B01J 37/08; B01J 37/14; B01J 37/16; B01J 37/34; C01B 3/40; C07C 1/12; C07C 2523/04; C07C 2523/50

(57) ABSTRACT

27 The present invention refers to a process comprising 3-10 metal atoms atomic quantum clusters (AQCs) supported on metal oxides as catalysts for oxygen release; which can be applied to the oxidation of fuels. Additionally, the present invention refers to the use of said 3-10 metal atoms AQCs supported on metal oxides as oxygen carriers in chemical looping reactions, and to catalysts and chemical compositions comprising said 3-10 metal atoms AQCs supported on metal oxides.

### TECHNICAL FIELD OF THE INVENTION

The present invention relates to the field of chemical looping (CL) technology and, more particularly, to oxygen carriers based on metal oxide supported atomic quantum clusters (AQCs) and application thereof in chemical-looping processes.

### BACKGROUND OF THE INVENTION

Global warming and climate change are most likely linked to the increasing concentration of the greenhouse gas carbon dioxide (CO₂) in the atmosphere. Therefore, it is imperative to develop and implement processes that avoid the emission of anthropogenic CO₂. One possible midterm solution is carbon-dioxide capture and storage (CCS).

When looking for a promising technology for reducing global CO₂ emissions, the so-called chemical-looping combustion (CLC) process, an emerging 3rd-generation CCS technology, is particularly attractive due to its very low predicted CO₂-capture costs compared to the currently available technology. CLC involves the use of an oxygen carrier that transfers oxygen from the air to the fuel, avoiding direct contact between them. A CLC system typically comprises an air and a fuel reactor. In the fuel reactor lattice oxygen from the solid-state oxygen carrier is used to combust a hydrocarbon fuel, which yields, after the condensation of steam, a pure stream of CO₂ suitable for sequestration. In the air reactor, a stream of air is used to regenerate the oxygen carrier material.

Although chemical looping technology has been predominantly developed for the so-called chemical looping combustion (CLC) of fuels such as coal or natural gas, more recently the focus of chemical looping is shifting towards the production of hydrogen and other chemicals. In this way, products with higher added value can be generated. With this technology, when the focus is on the production of chemicals, oxygen carrier materials are regenerated by other oxidizing agents instead of air, such as CO₂ or even H₂O, with respective productions of CO and H₂. Therefore, the chemical looping technology promises to be a transformational way for clean and efficient fuel conversion to electricity, hydrogen, and syngas.

A key aspect in this process is to find suitable solid-state oxygen carriers, with metal oxides being the most commonly used materials. The temperature and oxygen partial pressure under which oxide materials will react are controlled by their thermodynamic equilibria with respect to reduction and oxidation. This can be done using the Ellingham diagram for the processes of interest (Annu. Rev. Chem. Biomol. Eng. 2015. 6:3.1-3.23).

However, besides having such suitable redox thermodynamics, an ideal material must meet several other requirements in terms of kinetics, durability, etc. As an example, a good candidate from the thermodynamic point of view is MnO₂/Mn₂O₃ with equilibrium temperature at 730 K in air. However, reoxidation from Mn₂O₃ to MnO₂ is extremely slow, rendering the material inappropriate as an oxygen carrier (Energy Environ. Sci., 2017,10, 818-831).

Therefore, there remains a strong need in the art to develop suitable oxygen carrier materials with high oxygen-carrying capacity, good kinetics and versatility for more efficient chemical looping technology applications.

### FIGURES

FIG. 1. Diffuse reflectance spectroscopy (DRS) of Ag₅@CeO₂ after treatment with O₂ and after treatment with He.

FIG. 2. DRS of Ag₅@CeO₂ after treatment with O₂ (left) and after treatment with H₂ (right).

FIG. 3. XPS of CeO₂ in Cu₅@CeO₂ before (a) and after (b) Ar and light treatment at 450° C.

FIG. 4. Hydrogen production with Cu₅@CeO₂ in different reduction and oxidation cycles measured by GC-TCD at 450° C.

FIG. 5. (a) GC-TCD detection of CO and H₂ during vacancy formation with Ar:CH₄ (2:1) and light and (b) mass spectrometry detection of CH₄ and CO₂ during vacancy elimination with Ar:CO₂ (2:1) in the dark for Cu₅@CeO₂.

FIG. 6. shows a schematic illustration of a hydrogen production process according to an embodiment of the present invention.

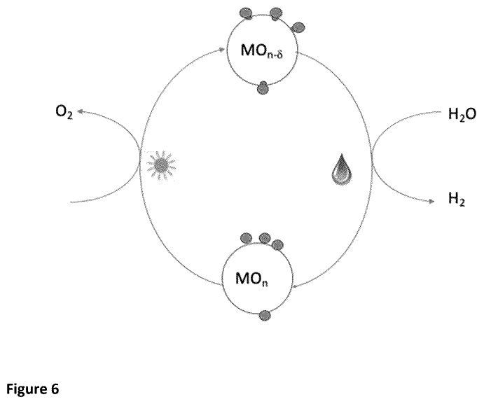

FIG. 7 shows the rate of hydrogen production per gram of Cu5@CeO₂ catalyst in the process of Example 6.

FIG. 8 shows the rate of hydrogen production per gram of Cu5@LSM 35 catalyst in the process of Example 7.

FIG. 9 shows the rate of hydrogen production per gram of Ag5@CeO₂ catalyst in the process of Example 8.

### BRIEF DESCRIPTION OF THE INVENTION

The authors of the present invention have surprisingly found that clusters of small atomicity deposited onto different metal oxides, drastically reduce the activation energy for both oxidation and reduction reactions involved in chemical looping, providing a new catalytic way to either reduce the temperature of the processes involved or improve the kinetics at the same temperature used for the processes without clusters.

Advantageously, the possibility to reduce the temperature of the chemical looping processes can additionally help to increase the oxygen carrier capacity of the oxide, avoiding the deterioration of the oxides which occurs at high temperatures and leads to a number of vacancies in a much lower level than the theoretical one. This reduces the amount of oxygen that an oxygen carrier can support at lower temperatures.

This finding makes these catalysts suitable solid-state oxygen carriers for different chemical looping processes with a focus on the production of heat and chemicals.

Therefore, a first aspect of the present invention refers to a catalytic process comprising the steps of:
i. providing a catalyst comprising atomic quantum clusters (AQCs) consisting of 3 to 10 metal atoms and a metal oxide, wherein the AQCs are deposited on the surface of the metal oxide; and;
ii. contacting said catalyst with a reducing agent at a temperature between room temperature and 800° C., to release oxygen from the metal oxide;
iii. optionally contacting said catalyst with an oxidizing agent for at least partially replenishing the oxygen in the metal oxide;
wherein said reducing agent is selected from an inert atmosphere, a reductive atmosphere, or a mixture thereof.

A second aspect of the invention relates to an oxygen-deficient catalyst obtainable by a process as defined above.

A third aspect refers to a catalytic composition comprising:
a) a catalyst comprising atomic quantum clusters (AQCs) consisting of 3 to 10 metal atoms and a metal oxide, wherein the AQCs are deposited on the surface of the metal oxide; and
b) a reducing agent selected from an inert atmosphere, a reductive atmosphere or a mixture thereof.

A fourth aspect relates to the use of a catalyst comprising AQCs consisting of 3 to 10 metal atoms and a metal oxide, wherein the AQCs are deposited on the surface of the metal oxide as oxygen carriers for chemical looping reactions.

These aspects and preferred embodiments thereof are additionally also defined hereinafter in the detailed description, as well as in the claims.

### DETAILED DESCRIPTION OF THE INVENTION

Unless defined otherwise, all technical and scientific terms used herein have the same meaning as commonly understood to one of ordinary skill in the art to which this disclosure belongs.

As defined above, in a first aspect, the present invention relates to a catalytic process comprising the steps of:
i. providing a catalyst comprising atomic quantum clusters (AQCs) consisting of 3 to 10 metal atoms and a metal oxide, wherein the AQCs are deposited on the surface of the metal oxide; and;
ii. contacting said catalyst with a reducing agent at a temperature between room temperature and 800° C., to release oxygen from the metal oxide;
iii. optionally contacting said catalyst with an oxidizing agent for at least partially replenishing the oxygen in the metal oxide;
wherein said reducing agent is selected from an inert atmosphere, a reductive atmosphere, or a mixture thereof.

In the process of the invention, the metal oxide of the catalyst donates lattice ◯ atoms upon contact with a reducing agent leading to the formation of an oxygen-deficient metal oxide (MO_{x-δ},), i. e., a metal oxide in a reduced or less oxidized state, through the creation of oxygen vacancies (OVs). This may result in (i) the release of O₂ from the lattice; (ii) or the partial oxidation of the oxygen to create peroxides; (iii) or the oxidation of a reducing agent (for example, hydrogen or fuels) taking O²⁻ from the lattice. The reduced metal oxide may then be partially or totally oxidized back to its original state (MOₓ) by a step in which the reduced metal is re-oxidized in the presence of an appropriate agent which provides oxygen to be incorporated into the metal oxide eliminating partially, or totally, the oxygen vacancies.

### Step (i)

The process of the present invention comprises a step (i) of providing a catalyst comprising metal Atomic Quantum Clusters (AQCs) having between 3 and 10 metal atoms and a metal oxide, wherein the AQCs are deposited on the surface of the metal oxide.

The term “Atomic Quantum Cluster”, abbreviated as AQC, is understood as metal Atomic Quantum Cluster. Metal AQCs are formed exclusively by zero-oxidation-state metal atoms, Mₙ, with less than 500 metal atoms (Mₙ, n<500), and with a size of less than 2 nm. The AQCs are stable over time. In addition, AQCs are known for no longer behaving like a “metal” and their behavior becomes molecular like. Therefore, new properties which are not observed in the nanoparticle, microparticle or bulk metallic forms appear in these clusters, as reported in EP1914196A1. Therefore, the physical-chemical properties of the AQCs cannot be simply extrapolated from those of the nano/microparticles.

The AQCs according to the present invention consist of 3 to 10 metal atoms, preferably the metal AQCs consist of 4 to 8, 5 to 7 metal atoms.

In a preferred embodiment, the metal AQCs are monodisperse metal AQCs, such as monodisperse metal AQCs having 3, 4, 5, 6, 7, 8, 9 or 10 metal atoms. As used herein, a monodisperse population is one in which the size of the metal clusters is identical as can be determined by currently used characterization procedures such as mass spectrometry. Preferably, the metal AQCs are monodispersed metal AQCs having 3, 4 or 5 metal atoms, more preferably, the metal AQCs are monodispersed metal AQCs having 5 metal atoms.

Thus, in a preferred embodiment, the metal AQCs consist of 3, 4, 5, 6, 7, 8, 9 or 10 metal atoms, more preferably the metal AQCs consist of 3, 4 or 5 metal atoms, most preferably the metal AQCs consist of 5 metal atoms.

Throughout the specification, unless the context requires otherwise, the term “consisting of”, and variations such as “consists of”, will be understood to imply the inclusion of a stated integer, step, group of integers or group of steps, with the exclusion of any other integer, step, group of integers or group of steps. Thus, it will be understood that references to AQCs consisting of “n” metal atoms, wherein n is an integer, refer to AQCs with only “n” transition metal atoms.

The metals of the AQCs suitable in the process of the present invention are transition metals, preferably transition metals selected from Ag, Cu, Au, Pt, Pd, Fe, Co, Ni or their bi-metal and multi-metal combinations, more preferably are selected from Pt, Ag, Cu or their bi-metal and multi-metal combinations. In a preferred embodiment, the metal is Cu. In another preferred embodiment, the metal is Pt. In a further preferred embodiment, the metal is Ag.

The metal AQCs suitable in the process of the present invention can be obtained by any suitable means known in the art. As a non-limiting example, methods for the preparation of metal AQCs are disclosed in S. Huseyinova et al., J. Phys. Chem. C, 2016, 120, 15902-15908 or V. Porto et al. Adv. Funct. Mater. 2022, 2113028 (1-14).

As a non-limiting example, the catalysts according to the present invention can be obtained by impregnating a solution of metal AQCs onto the metal oxide, and drying the obtained mixture. For example, the catalysts can be prepared by conventional methods known in the art such as those disclosed in V. Meille et al, Appl. Catal. A Gen. 2006, 315, 1-17.

In a particular embodiment, the catalysts according to the present invention may be subjected to an activation treatment prior to step i). For example, the catalysts may be subjected to calcination under a reductive atmosphere. Preferably, calcination may be performed at a temperature of 500-600° C. for about 1 hour under an Ar/H₂ flow.

The metal oxide of the present invention can comprise any metal oxide known in the art suitable as oxygen carrier in chemical looping reactions. Non-limiting examples of metal oxides typically used in chemical looping include pure metal oxides such CuO, CdO, NiO, Mn₂O₃, Fe₂O₃, and CoO; as well as mixed oxides; perovskite-type oxides; and substituted perovskite-type oxides.

In a particular embodiment, the metal oxide is selected from the group consisting of metal oxides comprising a rare earth element, preferably La, Ce, Pr, Nd, Pm, Sm, Eu, Gd, or mixtures thereof; such as La₂O₃, CeO₂, Ce₂O₃, Ce₃O₄, Nd₂O₃, Sm₂O₃, Eu₂O₃, Gd₂O₃, or mixtures thereof.

In another particular embodiment, the metal oxide may be a perovskite-type metal oxide. In a more particular embodiment, the metal oxide is a perovskite-type metal oxide of formula ABO₃, in which the A and B sites may have a configuration of A1₁₋ₓA2ₓ and/or B1_{1-y}B2_{y}; where A comprises at least a rare earth element and B is at least one transition metal element. In a preferred embodiment, A is at least a rare earth element selected from the group consisting of La, Ce, Pr, Nd, Pm, Sm, Eu, Gd, and combinations thereof. In another preferred embodiment, B is at least one transition metal element selected from the group consisting of Cr, Mo, Mn, Fe, Ru, Co, Ni and Cu. Preferably, the perovskite-type metal oxide is selected from LaMnO₃, La₁₋ₓSrₓMnO₃ and La₁₋ₓCeₓMnO₃. More preferably, the perovskite-type metal oxide is La_{0.65}Sr_{0.35}MnO₃.

In a further particular embodiment, the metal oxide may be a metal oxide comprising a transition metal element selected from Cr, Mo, Mn, Fe, Ru, Co, Ni, Cu, or mixtures thereof, such as MnO₂, Mn₂O₃, Co₃O₄, Fe₂O₃, Fe₃O₄, CoFe₂O₄, NiFe₂O₄, ZnFe₂O₄, or mixtures thereof.

In an embodiment, the metal oxide is selected from the group consisting of TiO₂, WO₃, MnO₂, Mn₂O₃, Co₃O₄, Fe₂O₃, Fe₃O₄, CoFe₂O₄, NiFe₂O₄, ZnFe₂O₄, FeAl₂O₄, BiFeO₃, FeAl₂O₄, CoAl₂O₄, La₂O₃, CeO₂, Ce₂O₃, Ce₃O₄, Nd₂O₃, Sm₂O₃, Eu₂O₃, Gd₂O₃, LaMnO₃, La₁₋ₓSrₓMnO₃ and La₁₋ₓCeₓMnO₃, or combinations thereof.

In a preferred embodiment, the metal oxide is selected from CeO₂, Ce₂O₃, Ce₃O₄, MnO₂, Mn₂O₃, Co₃O₄, BiFeO₃, La₁₋ₓSrₓMnO₃ and La₁₋ₓCeₓMnO₃ or a combination thereof. In a more preferred embodiment, the metal oxide is a cerium oxide, even more preferably the metal oxide is CeO₂, most preferably the metal oxide is La_{0.65}Sr_{0.35}MnO₃.

In a particular embodiment, the metal oxide is in any form that maximizes its surface area, preferably it can be in particulate form or powdered form. In a particular embodiment, the metal oxide can be in the form of nanostructures, i.e. having at least one dimension equal to or less than 1000 nm, such as nanopowders, nanowires, nanorods, nanoparticles, nanoplatelets or mixtures thereof. In a preferred embodiment, the average particle size of the nanostructures is between 10 and 200 nm.

In a particular embodiment, the metal oxide has a surface area between 1 square meter per gram and 2000 square meter per gram, preferably between 5 and 1000, more preferably between 10 and 500 square meter per gram, even more preferably between 10 and 100 square meter per gram, most preferably between 20 and 80 square meter per gram.

In a particular embodiment, 0.01% to 20% of the surface area of the metal oxide is covered with the AQCs, preferably 0.1% to 10% of the surface area, more preferably 0.5% to 5% of the surface area of the metal oxide is covered with the AQCs.

In a particular embodiment, the loading of AQCs in the catalyst ranges between a 0.01 and a 10% wt, preferably between 0.1 and 5% wt based on the total weight of the catalyst.

Additionally, or alternatively, metal oxides can be defined as volatile or non-volatile metal oxides. In the context of the present invention, the term “non-volatile metal oxide” refers to a metal oxide species that remains in a solid phase throughout the process. Non-limiting examples of non-volatile metal oxides include titanium oxide, cerium oxides, iron oxides, cobalt oxides, hercynites or perovskites.

For volatile oxides, the temperature required for reduction is greater than the vaporization temperature of the metal oxide, thereby causing it to undergo a phase transition during the high temperature thermal reduction step. Preferably, the metal oxide of the invention is a non-volatile metal oxide.

In a particular embodiment, the catalyst comprises Cu AQCs deposited on the surface of CeO₂. Preferably, the Cu AQCs consist of 5 metal atoms.

In a preferred embodiment, the catalyst comprises Ag AQCs deposited on the surface of CeO₂. Preferably, the Ag AQCs consist of 5 metal atoms.

In another preferred embodiment, the catalyst comprises Cu AQCs consisting of 5 metal atoms deposited on the surface of La_{0.65}Sr_{0.35}MnO₃.

### Step (ii)

The process of the present invention further comprises a step (ii) of contacting the catalyst of step (i) with a reducing agent to release oxygen from the metal oxide of the catalyst.

In the context of the present invention, by “release oxygen from the metal oxide” it is meant the reduction of the metal oxide through the creation of oxygen vacancies (OVs), with the concomitant release of O₂ from the lattice; partial oxidation of the oxygen to create peroxides; or the oxidation of the reducing agent taking O²⁻ from the lattice.

The expression “contacting” of step (ii) refers to a physical contact between the catalyst and the reducing agent. The contacting of step (ii) can be performed by any suitable means. In a preferred embodiment, the contact of step (ii) can be achieved by passing a flow of a reducing agent in gaseous form through the catalyst.

In the context of the present invention, by reducing agent it is meant an atmosphere suitable to promote removal of oxygen from the catalyst. The reducing agent suitable for the present invention can be an inert atmosphere, a reductive atmosphere or a mixture thereof.

As used herein, the term “inert atmosphere” refers to vacuum; a partial pressure of oxygen lower than the thermodynamic partial pressure needed to achieve a negative value of AG (Gibbs free energy) for the vacancy formation at the temperature of the process, preferably a partial pressure of oxygen less than 1 bar, more preferably less than 0.1 bar; or an inert gas atmosphere. As used herein, the term “reductive atmosphere” refers to an atmosphere comprising a reductive gas.

Therefore, in a particular embodiment, the reducing agent is selected from the group consisting of vacuum; a partial pressure of oxygen of less than 1 bar, preferably less than 0.1 bar; an inert gas; a reductive gas; or a combination thereof. Preferably, the reducing agent is an inert gas, a reductive gas or a combination thereof. In a more particular embodiment, the inert gas is selected from helium, nitrogen, argon and mixtures thereof. In another more particular embodiment, the reductive gas is selected from hydrogen, methane, low carbon fuels (C2-C5), carbon monoxide and mixtures thereof. In a particular embodiment, the reductive gas may be derived from a feedstock such as coal or biomass.

In some particular embodiments, the reducing agent of step (ii) reacts with at least part of the oxygen released from the catalyst, thus forming an oxidized by-product. In a particular embodiment, the reducing agent may undergo a complete, partial or selective oxidation. In a particular embodiment, the reducing agent of step (ii) is hydrogen and the oxidized by-product is water. In another particular embodiment, the reducing agent of step (ii) is methane and the oxidized by-product is carbon dioxide, carbon monoxide, syngas or a mixture thereof.

In a particular embodiment, the hourly space velocity (GHSV) of the reducing agent over the catalyst in step (ii) is between 10 h⁻¹ and 1000000 h⁻¹, preferably between 1000 and 500000 h⁻¹, more preferably between 10000 and 100000 h⁻¹. GHSV may be defined as the volume of gas-typically expressed at standard conditions-entering a reactor per hour per unit volume of catalyst.

In a particular embodiment, step (ii) is performed for a time of between 1 second and 24 hours, preferably between 10 seconds and 10 hours, more preferably between 30 seconds to 2 hours. In a particular embodiment, step (ii) is performed for a time of about 30 seconds, about 1 minute, about 5 minutes, about 10 minutes, about 30 minutes, about 1 hour, about 2 hours, about 4 hours, about 8 hours, about 16 hours, about 24 hours. In a preferred embodiment, step (ii) is performed for a time of about 5 minutes.

Step (ii) is performed at a temperature ranging from room temperature to 800° C., preferably at a temperature ranging from 200 to 700° C., preferably between 350 and 600° C., more preferably at a temperature of about 550° C.

In another preferred embodiment, step (ii) is performed at room temperature.

The term “room temperature” in the context of the present invention means that the temperature is between 10° C. and 40° C., preferably between 15° C. and 30° C., more preferably between 20° C. and 25° C.

In a preferred embodiment, step (ii) is performed at a temperature of 250 to 600° C. and the reducing agent is an inert gas selected from argon, helium or a mixture thereof.

In another preferred embodiment, step (ii) is performed at a temperature of 250 to 600° C. and the reducing agent is a reductive gas such as hydrogen, methane or a mixture thereof.

In a particular embodiment, step (ii) is performed under a pressure ranging between 0.1 bar and 100 bar. In a preferred embodiment, step (ii) is performed at atmospheric pressure.

In a particular embodiment, step (ii) further comprises irradiating the catalyst with light.

In a more particular embodiment, the light irradiated in step (ii) is derived from natural sunlight. In a further embodiment, the light irradiated in step (ii) is derived from an artificial light source such as a solar simulator lamp, a UV lamp or a Xe-lamp. In a particular embodiment, an artificial light source can be used in combination with natural sunlight.

In a particular embodiment, the light irradiated in step (ii) is selected from sunlight, visible light, UV light or a combination thereof. In a preferred embodiment the light is visible light.

In a particular embodiment, the irradiation of step (ii) is performed using a light flux from about 0.1 to 20 mW/cm², or from 0.5 to 10 mW/cm².

In a particular embodiment, concentrated solar energy is used to obtain at least part of the energy required to the irradiation of step (ii). In a particular embodiment, solar energy can be concentrated using solar concentrators such as tower and dish systems.

### Step (iii)

The process of the present invention may further comprise a step (iii) of contacting the catalyst with an oxidizing agent for at least partially replenishing the oxygen in the metal oxide. Therefore, in this step, if conducted, the oxygen deficient or reduced metal oxide is partially or totally oxidized back to its original state (MOₓ). As a result, the oxidizing agent is depleted of oxygen, possibly yielding a reduced product.

In a particular embodiment, the oxidizing agent can be selected from air, carbon dioxide, carbon monoxide, water, and combinations thereof.

The contacting of step (iii) can be performed by any suitable means. In a particular embodiment, the contacting of step (iii) can be performed by dispersing the catalyst in the oxidizing agent. In a preferred embodiment, the contacting of step (iii) is performed by passing a flow of the oxidizing agent through the catalyst.

In some particular embodiments, the oxidizing agent of step (iii) undergoes a reduction upon transferring oxygen to the catalyst, thus forming a reduced by-product. In a particular embodiment, the oxidizing agent of step (iii) is water, and hydrogen is formed as reduced by-product. In another particular embodiment, the oxidizing agent of step (iii) is carbon dioxide, and methane is formed as reduced by-product.

In a particular embodiment, the GHSV (hourly space velocity) of the oxidizing agent over the catalyst in step (iii) is between 10 h⁻¹ and 1000000 h⁻¹ preferably between 1000 and 500000 h⁻¹, preferably between 10000 and 100000 h⁻¹.

In a particular embodiment, step (iii) is performed for a time of between 1 second and 24 hours, preferably between 10 seconds and 10 hours, more preferably between 30 seconds to 2 hours. In a particular embodiment, step (iii) is performed for a time of about 30 seconds, about 1 minute, about 5 minutes, about 10 minutes, about 30 minutes, about 1 hour, about 2 hours, about 4 hours, about 8 hours, about 16 hours, about 24 hours. In a preferred embodiment, step (iii) is performed for a time of about 5 minutes.

In a particular embodiment, step (iii) is performed under a pressure ranging between 0.1 and 100 bar. In a preferred embodiment, step (iii) is performed at atmospheric pressure.

In some embodiments, step (iii) further comprises irradiating the catalyst with light.

In a more particular embodiment, the light irradiated in step (iii) is derived from natural sunlight. In a further embodiment, the light irradiated in step (iii) is derived from an artificial light source such as a solar simulator lamp, a UV lamp or a Xe-lamp. In a particular embodiment, an artificial light source can be used in combination with natural sunlight.

In a particular embodiment, the light irradiated in step (iii) is selected from sunlight, visible light, UV light or a combination thereof. In a preferred embodiment the light is visible light.

In a particular embodiment, the irradiation of step (iii) is performed using a light flux from about 0.1 to 20 mW/cm², or from 0.5 to 10 mW/cm².

In a particular embodiment, concentrated solar energy is used to obtain at least part of the energy required to the irradiation of step (iii).

In some alternative embodiments, step (iii) can be performed substantially in the absence of light. In a particular embodiment, step (iii) is performed in the total absence of light.

For the purposes of this disclosure, the term substantially in the absence of light in step (iii) means that the amount of light reaching the catalyst may lead to formation of oxygen in a maximum amount of 10 mol %, preferably 1 mol %, and more preferably 0.1 mol % relative to the total amount of the corresponding reduced by-product being produced during step (iii), such as the total amount of hydrogen or methane.

The oxidizing agent for the contacting of step (iii) can be in any suitable form such as liquid or gas phase.

In a particular embodiment, the oxidizing agent is water. In a more particular embodiment, the water is in the form of liquid water, water vapor or steam, or a combination thereof. In a preferred embodiment, the water is water vapor. In another particular embodiment, the oxidizing agent is carbon dioxide.

In an embodiment, step (iii) is performed at a temperature ranging from room temperature to 800° C., preferably at a temperature ranging from 200 to 700° C., preferably between 350 and 600° C., more preferably at a temperature of about 550° C.

In another preferred embodiment, step (iii) is performed at room temperature.

In a further embodiment, steps (ii) and (iii) are performed in reverse order, i. e., firstly step (iii) and next step (ii).

In a particular embodiment, the process of the invention further comprises repeating steps (ii) and (iii) at least once for oxygen release and replenishment of the catalyst metal oxide lattice.

In a particular embodiment, steps (ii) and (iii) are repeated at least once, at least twice, at least 3 times, at least 5 times, at least 10 times, at least 50 times, at least 100 times, at least 1000 times, at least 10 000 times, at least 100 000 times. In a preferred embodiment, steps (ii) and (iii) are repeated at least 3 times. In a more preferred embodiment, steps (ii) and (iii) are repeated at least 100 times. In an even more preferred embodiment, steps (ii) and (iii) are repeated at least 1000 times.

In a particular embodiment, steps (ii) and (iii) are performed in reverse order, i. e. first step (iii) and next step (ii), wherein steps (iii) and (ii) are repeated at least once, at least twice, at least 3 times, at least 5 times, at least 10 times, at least 50 times, at least 100 times, at least 1000 times, at least 10 000 times, at least 100 000 times. In a preferred embodiment, steps (iii) and (ii) are repeated at least 3 times. In a more preferred embodiment, steps (iii) and (ii) are repeated at least 100 times. In an even more preferred embodiment, steps (iii) and (ii) are repeated at least 1000 times.

In a preferred embodiment the process of the invention comprises the steps of:
i. providing a catalyst comprising atomic quantum clusters (AQCs) consisting of 3 to 10 metal atoms and a metal oxide, wherein the AQCs are deposited on the surface of the metal oxide;
ii. contacting said catalyst with CH₄ at a temperature between room temperature and 800° C. to release oxygen from the metal oxide, preferably while irradiating said catalyst with light, wherein hydrogen and CO are formed as oxidized by-products; and
iii. contacting said catalyst with CO₂ substantially in the absence of light for at least partially replenishing the oxygen in the metal oxide, wherein CH₄ is formed as a reduced by-product.

In another preferred embodiment the process of the invention comprises the steps of:
i. providing a catalyst comprising atomic quantum clusters (AQCs) consisting of 3 to 10 metal atoms and a non-volatile metal oxide, wherein the AQCs are deposited on the surface of the non-volatile metal oxide;
ii. contacting said catalyst with an inert atmosphere at a temperature between room temperature and 800° C. to release oxygen from the metal oxide, preferably while irradiating said catalyst with light; and
iii. contacting said catalyst with water substantially in the absence of light for at least partially replenishing the oxygen in the metal oxide and produce hydrogen.

Within this particular embodiment, the term substantially in the absence of light in step (iii) means that the amount of light reaching the catalyst may lead to formation of oxygen in a maximum amount of 10 mol %, preferably 1 mol %, and more preferably 0.1 mol % relative to the total amount of hydrogen being produced.

In a more preferred embodiment, steps (ii) and (iii) are performed in reverse order.

In another more preferred embodiment, steps (ii) and (iii) are repeated at least once, preferably at least 3 times, even more preferably at least 100 times.

It is further envisaged that the procedure can suitably use the low-energy part of the solar spectrum to reach and maintain the temperatures needed for the whole process. In a particular embodiment, the low-energy part of the solar spectrum may be infrared light below 0.5 eV, preferably below 0.1 eV.

In a particular embodiment, concentrated solar energy is used to obtain at least part of the energy required to reach the temperature of step(s) (ii) and/or (iii). In a further embodiment, concentrated solar energy is used to obtain at least part of the energy required to the irradiation of step (ii). In a particular embodiment, solar energy can be concentrated using solar concentrators such as tower and dish systems.

As explained above, metal AQCs supported on metal oxides have surprisingly high conversion efficiency even at low temperatures. This is an important advantage over prior art processes since it avoids deterioration of the oxides which occurs at high temperatures. Therefore, a higher oxygen carrier capacity of the catalyst is achieved together with a higher durability in successive cycle reactions.

### Catalyst

In a second aspect, the present invention relates to an oxygen-deficient catalyst obtainable by a process as defined in the first aspect in any of its particular and preferred embodiments.

In the context of the present invention, the term “oxygen deficient” in relation to a catalyst refers to an excess of metal and/or absence of oxygen in a metal oxide with respect to a stoichiometric composition. As such, any metal oxide that has a higher atomic concentration ratio of its metal to oxygen than the ideal stoichiometric composition of the metal oxide is considered to be oxygen deficient and, therefore, has an excess of oxygen vacancies greater than 0%.

In a particular embodiment, the catalyst has an excess of oxygen vacancies of at least 0.1%, preferably at least 1%, more preferably at least 5%, even more preferably at least 10% relative to the stoichiometric composition of the metal oxide. In another particular embodiment the catalyst has an excess of oxygen vacancies between 0.1 and 100%, preferably between 1 and 80% and more preferably between 10 and 60% relative to the stoichiometric composition of the metal oxide, as may be determined by techniques well-known in the art such as Raman spectroscopy or XPS (X-ray photoelectron spectroscopy).

In a particular embodiment, the metal AQCs are monodisperse metal AQCs, preferably monodisperse metal AQCs having 3, 4 or 5 metal atoms, more preferably monodisperse metal AQCs having 5 metal atoms.

In a preferred embodiment, the oxygen deficient catalyst comprises Cu or Ag AQCs, preferably consisting of 5 metal atoms. In a more preferred embodiment, the metal oxide is CeO₂.

In a preferred embodiment, the oxygen deficient catalyst comprises Cu or Ag AQCs consisting of 5 metal atoms deposited on the surface of CeO₂.

### Composition

A third aspect refers to a catalytic composition comprising:
a) a catalyst comprising atomic quantum clusters (AQCs) consisting of 3 to 10 metal atoms and a metal oxide, wherein the AQCs are deposited on the surface of the metal oxide; and
b) a reducing agent selected from an inert atmosphere, a reducing atmosphere or a mixture thereof.

In a particular embodiment, the catalyst has an excess of oxygen vacancies of at least 0.1%, preferably at least 1%, more preferably at least 5%, even more preferably at least 10%, relative to the stoichiometric composition of the metal oxide. In another particular embodiment the catalyst has an excess of oxygen vacancies between 0.1 and 100%, preferably between 1 and 80%, and more preferably between 10 and 60% relative to the stoichiometric composition of the metal oxide.

In a particular embodiment, the metal AQCs are monodisperse metal AQCs, preferably monodisperse metal AQCs having 3, 4 or 5 metal atoms, more preferably monodisperse metal AQCs having 5 metal atoms.

In a preferred embodiment, the catalyst of the composition comprises Cu or Ag AQCs, preferably consisting of 5 metal atoms. In a more preferred embodiment, the metal oxide is CeO₂.

In a most preferred embodiment, the catalyst of the composition comprises Cu or Ag AQCs consisting of 5 metal atoms deposited on the surface of CeO₂.

In a particular embodiment, the reducing agent of the composition is an inert gas. Preferably, the inert gas is selected from argon, helium, nitrogen or a mixture thereof.

In another particular embodiment, the reducing agent is a reductive gas. Preferably, the reductive gas is selected from H₂, CO, methane or a mixture thereof.

Further particular and preferred embodiments for the catalyst and the reducing agent in the composition are as those described above for the previous aspects.

### Use

A fourth aspect relates to the use of a catalyst comprising AQCs consisting of 3 to 10 metal atoms and a metal oxide, wherein the AQCs are deposited on the surface of the metal oxide as oxygen carriers for chemical looping reactions.

The invention further relates to the use of an oxygen-deficient catalyst or a catalytic composition as defined in the second and third aspects in any of its particular and preferred embodiments for chemical-looping reactions.

In a particular embodiment, the chemical-looping reaction is selected from the group consisting of chemical looping combustion, syngas production, water-gas shift reaction, steam methane reforming, selective oxidation of hydrocarbons, hydrogen generation, photo/thermochemical water-splitting, thermochemical water-splitting, thermochemical carbon dioxide splitting. In a preferred embodiment, the use is for thermochemical water-splitting or photo/thermochemical water-splitting.

### Other Embodiments

1. An embodiment refers to a process for hydrogen production comprising the steps of:
   i. providing a catalyst comprising atomic quantum clusters (AQCs) consisting of 3 to 10 metal atoms and a non-volatile metal oxide, wherein the AQCs are deposited on the surface of the non-volatile metal oxide;
   ii. contacting said catalyst with water substantially in the absence of light to produce hydrogen; and
   iii. irradiating said catalyst with light in an inert atmosphere to form oxygen vacancies (OVs) with the subsequent release of oxygen from the metal oxide; and
   iv. optionally repeating steps ii) and iii) at least once for the production of hydrogen and release of oxygen;
   wherein steps ii) and iii) can be performed in reverse order.
2. Another embodiment refers to the process according to embodiment 1, wherein the AQCs consist of 5 metal atoms.
3. Another embodiment refers to the process according to embodiments 1 or 2, wherein the metal of the AQCs is selected from Ag, Cu, Au, Pt, Pd, Fe, Co, Ni or their bi-metal and multi-metal combinations.
4. Another embodiment refers to the process according to any of embodiments 1 to 3, wherein the metal of the AQCs is Cu, Ag, Pt or their bi-metal and multi-metal combinations.
5. Another embodiment refers to the process according to any of embodiments 1 to 4, wherein the loading of the AQCs deposited onto the surface of the non-volatile metal oxide is of between 0.01% and 20% of surface coverage.
6. Another embodiment refers to the process according to any of claims 1 to 5, wherein the non-volatile metal oxide is selected from TiO₂, WO₃, MnO₂, Mn₂O₃, Co₃O₄, Fe₂O₃, Fe₃O₄, CoFe₂O₄, NiFe₂O₄, ZnFe₂O₄, FeAl₂O₄, BiFeO₃, FeAl₂O₄, CoAl₂O₄, La₂O₃, CeO₂, Ce₂O₃, Ce₃O₄, Nd₂O₃, Sm₂O₃, Eu₂O₃, Gd₂O₃, LaMnO3, La₁₋ₓSrₓMnO₃ and La₁₋ₓCeₓMnO₃, or a combination thereof.
7. Another embodiment refers to the process according to any of embodiments 1 to 6, wherein the non-volatile metal oxide has a surface area between 1 square meter per gram and 2000 square meter per gram.
8. Another embodiment refers to the process according to any of embodiments 1 to 7, wherein step(s) (ii) and/or (iii) is/are performed at a temperature of between room temperature and 800° C., preferably between 350 and 600° C., more preferably at a temperature of about 550° C.
9. Another embodiment refers to the process according to any of embodiments 1 to 8, wherein step (ii) is performed for a time of between 1 second to 24 hours, preferably 30 seconds to 2 hours, more preferably about 5 minutes.

10. Another embodiment refers to the process according to any of embodiments 1 to 9, wherein step (iii) is performed for a time of between 1 second to 24 hours, preferably 30 seconds to 2 hours, more preferably about 5 minutes.
11. Another embodiment refers to the process according to any of embodiments 1 to 10, wherein the water is in the form of liquid water, water vapor or steam, or a combination thereof, preferably is water vapor.
12. Another embodiment refers to the process according to any of embodiments 1 to 11, wherein the light irradiated in step (iii) is sunlight, visible light, UV light or a combination thereof.
13. Another embodiment refers to the process according to any of embodiments 8 to 12, wherein solar concentrators are used to obtain at least part of the energy required to reach the temperature of step(s) (ii) and/or (iii), and/or the irradiation of step (iii).
14. Another embodiment refers to the process according to any of embodiments 1 to 13, comprising the steps of:
   i) providing a catalyst comprising Ag or Cu AQCs consisting of 5 metal atoms deposited on the surface of CeO₂ or La_{0.65}Sr_{0.35}MnO₃, respectively;
   ii) contacting said catalyst with water vapor substantially in the absence of light at a temperature of 350 to 600° C. to produce hydrogen,
   iii) contacting said catalyst with an inert atmosphere while irradiating said catalyst with visible light to release oxygen form the metal oxide,
   iv) optionally repeating steps ii) and iii) at least once for production of hydrogen and release of oxygen.
15. Another embodiment refers to the use of a catalyst comprising AQCs consisting of 3 to 10 metal atoms and a non-volatile metal oxide, wherein the AQCs are deposited on the surface of the non-volatile metal oxide for thermocatalytic hydrogen production and/or thermocatalytic water-splitting, and/or photocatalytic/thermocatalytic hydrogen production.

### EXAMPLES

The present invention will now be described by way of examples which serve to illustrate the construction and testing of illustrative embodiments. However, it is understood that the present invention is not limited in any way to the examples below.

### Example 1. Catalyst Preparation

Typically, 0.1 g of CeO₂ or La_{0.65}Sr0.35MnO₃ (purchased from Alfa Aesar) metal oxide were impregnated with 400 μL or 2 mL of metal Cu or Ag AQCs (synthesized and characterized according to the procedure previously reported on S. Huseyinova et al., J. Phys. Chem. C, 2016, 120, 15902-15908 or V. Porto et al. Adv. Funct. Mater. 2022, 2113028 (1-14) and Process for producing atomic quantum clusters, Application No. EP 18382038.0; Methods of preparing purified atomic quantum clusters, Application No. PCT/ES20 19/070403) with appropriate concentration to obtain the desired loading.

For Examples 2-5, the resulting solution was then dried in air in an incubator at 25° C. for 48 h, followed by a drying step at 80° C. overnight at 80 mbar.

For Examples 6-8, the resulting solution was dried at air overnight, followed by a calcination step at 550° C. for 1 h under an Ar/H₂ flow (9:1 in volume).

### Example 2. Vacancy Formation Cycles with He in the Dark

100 mg of Ag₅-CeO₂ catalyst with a loading of 0.5% wt were placed inside a gas-phase reactor. The reactor was heated to 550° C. at P=1.1 bar. A He flow was introduced into the reactor at a rate of 20 mL min⁻¹ for 1 hour to form vacancies in the metal oxide. This was followed by the introduction of a 20% O₂ in He flow into the reactor at a rate of 20 mL min⁻¹ for 1 hour to remove oxygen vacancies by replenishing the oxygen in the catalyst.

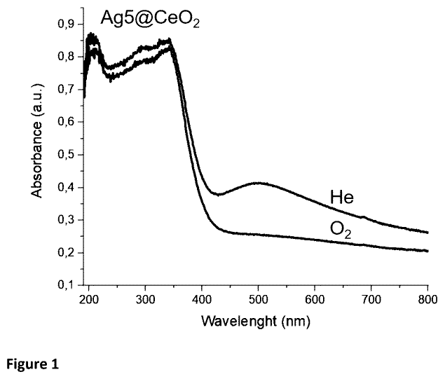

A colour change was observed in the catalyst material indicating the presence or absence of O₂ vacancies in the metal oxide. The formation of oxygen vacancies could also be determined by diffuse reflectance spectroscopy (DRS), by the presence of a band at =514 nm indicating O₂ vacancies on the CeO₂ and its disappearance after treatment with O₂ as can be observed in FIG. 1.

### Example 3. Vacancy Formation Cycles with H₂ in the Dark

100 mg of Ag₅-CeO₂ catalyst with a loading of 0.5% wt were placed inside a gas-phase reactor. The reactor was heated to 550° C. at P=1.1 bar. A first treatment with a flow of 20% O₂ in He was performed (dV/dt=20 mL min⁻¹) for 1 hour in order to eliminate any oxygen vacancies present in the initial sample. Then, a step of new vacancy formation was carried out by the introduction of a H₂ flow for 90 min.

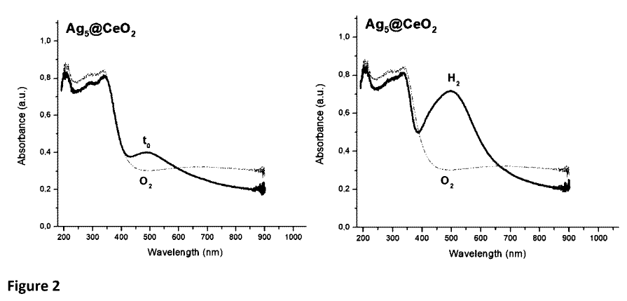

DRS showed the elimination of initial vacancies (t₀) in the sample created to some extent during the process of deposition (left) and the formation of new vacancies with H₂ (right), as evidenced by the presence or absence of the band at =514 nm shown in FIG. 2.

### Example 4. Vacancy Formation with Ar and Light Followed by H₂ Production with Water

50 mg of Cu₅-CeO₂ catalyst with a loading of 0.02% wt were placed inside a gas-phase reactor. The reactor was heated to 450° C. at P=1.1 bar. An Argon flow was introduced into the reactor under light irradiation (Airradiation=19.6 cm², dV/dt=10 mL min⁻¹, φ=30%, Irradiation source: 200 W Hg (Xe) lamp) for 2 hours. This was followed by the introduction of water vapor into the reactor at a rate of 3 mL min⁻¹ carried by Ar using a saturator (volume fraction φ=30%) without light irradiation for 1 hour.

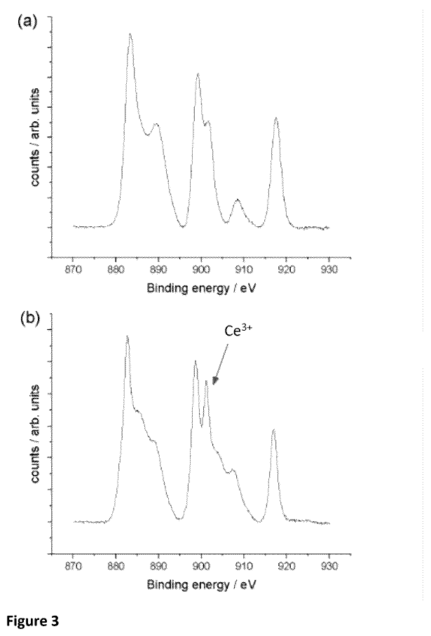

FIG. 3 shows the CeO₂ XPS characterization of the Cu₅@CeO₂ sample before (a) and after (b) Ar and light treatment at 450° C., where a Ce³⁺ signal increase can be observed due to vacancy formation in the metal oxide.

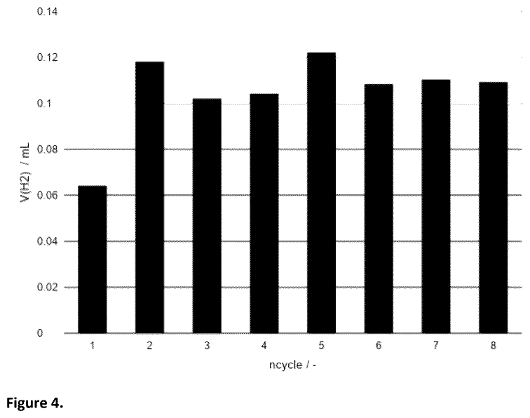

Cycles of Ar/light irradiation and H₂O/dark were repeated 8 times. Hydrogen evolution for each cycle was analyzed by GC-TCD as shown in FIG. 4.

### Example 5. Vacancy Formation With Ar:Ch₄ (2:1) and Light Followed by Ch₄ Production with CO₂

50 mg of Ag₅-CeO₂ catalyst with a loading of 1% wt were placed inside a gas-phase reactor. The reactor was heated to 500° C. at P=1.1 bar. An Ar:CH₄ (2:1) flow was introduced into the reactor under light irradiation (Airradiation=19.6 cm², dV/dt=10 mL min⁻¹, φ=30%, Irradiation source: 200 W Hg (Xe) lamp) for 0.5 hour. CH₄ was transformed in this step to CO+H₂. This was followed by introduction of CO₂ carried by Ar into the reactor at a rate of 20 mL min⁻¹ (Ar:CO₂, 2:1) in dark condition for 0.5 hour. CO2 was transformed in this step to CH₄.

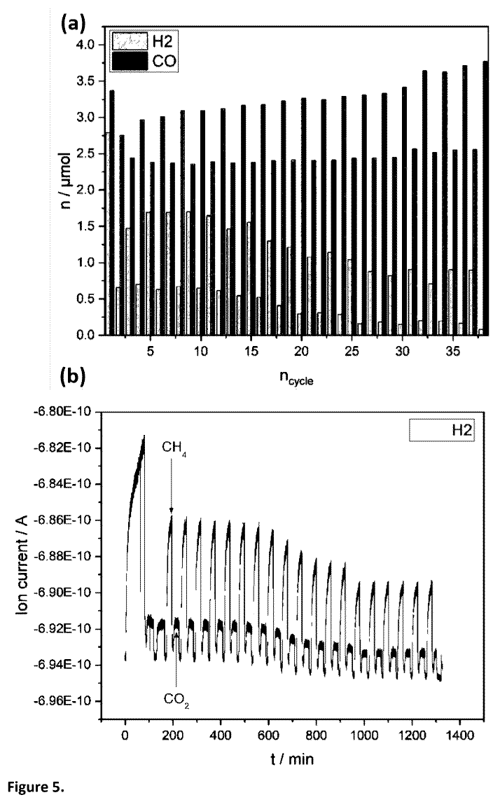

The cycles of CH₄/light and CO₂/dark were repeated 40 times. The detection of CO and H₂ during the vacancy formation cycles was analyzed by GC-TCD, as shown in FIG. 5a. The evolution of CH₄ and CO₂ as analyzed by mass spectrometry is shown in FIG. 5b.

### Example 6. Hydrogen Production Experiment using Cu5 supported on CeO₂ as Catalyst

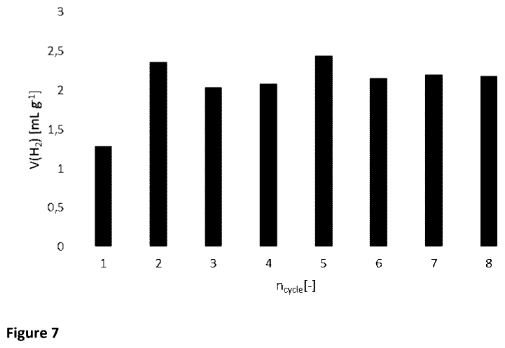

50 mg of Cu5-CeO₂ catalyst were dispersed in water and deposited on a 20 cm² area of a 30 cm² sinter plate, followed by placing the catalyst inside a gas-phase reactor. The reactor and its periphery were evacuated 3 times. Afterward, the whole setup was purged with Argon for 30 min to remove air. After heating the reactor to 450° C. in 2 h while purging with Argon, water vapor was introduced into the reactor at a rate of 3 mL min⁻¹ carried by Ar using a saturator (volume fraction φ=30%) for 1 hour. This step was followed by an irradiation step with a 200 W Xenon lamp under an Argon flow at a rate of 10 mL min⁻¹ for 2 hours. Hydrogen evolution was analyzed by a gas chromatograph [GC-2014 Shimadzu with following columns: Molsieb 13×60/80 and Porapak M 80/100]. The steps (ii) of water vapor introduction and (iii) light irradiation were repeated 7 additional times. In the 8 cycles performed, an average of 2 mL g⁻¹ H₂ were produced as shown in FIG. 7.

### Example 7. Hydrogen Production Experiment using Cu5 Supported on LSM 35 as Catalyst.

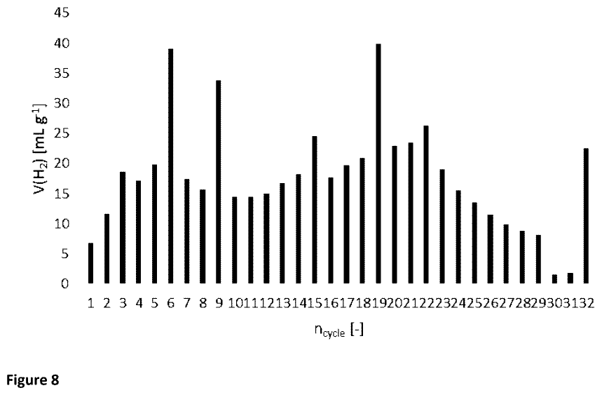

50 mg of Cu5-La_{0.65}Sr0.35MnO₃ catalyst were dispersed in water and deposited on a 20 cm² area of a 30 cm² sinter plate, followed by placing the catalyst inside a gas-phase reactor. The reactor and its periphery were evacuated 3 times. Afterward, the whole setup was purged with Argon for 30 min to remove air. After heating the reactor to 550° C. in 2.5 h while purging with Argon, water vapor was introduced into the reactor at a rate of 3 mL min⁻¹ carried by Ar using a saturator (volume fraction φ=30%) for 0.5 hours. This step was followed by an irradiation step with a 200 W Xenon lamp under an Argon flow at a rate of 10 mL min⁻¹ for 1 hour. Hydrogen evolution was analyzed by a gas chromatograph [GC-2014 Shimadzu with following columns: Molsieb 13×60/80 and Porapak M 80/100]. The steps (ii) of water vapor introduction and (iii) light irradiation were repeated 31 additional times. In the 32 cycles performed, in the first 23 cycles an average of 20 mL g⁻¹ H₂ were produced as shown in FIG. 8. After slowly decreasing to nearly 0 mL g⁻¹ H₂ per cycle (beginning at cycle 24), the catalyst was irradiated for 90 min with a 200 W Xenon lamp under Ar to get 20 mL g⁻¹ H₂ in the last cycle (32).

### Example 8. Hydrogen Production Experiment using Ag5 Supported on CeO₂ as Catalyst

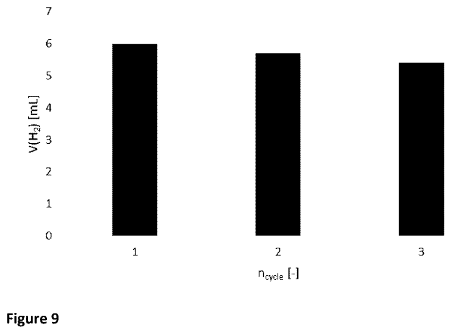

50 mg of Ag 5-CeO2 catalyst were dispersed in water and deposited on a 12.25 cm² steel plate, followed by placing the catalyst inside a gas-phase reactor. The reactor and its periphery were evacuated 3 times. Afterward, the whole setup was purged with Argon for 30 min to remove air. After heating the reactor to 500° C. for 1 h while purging with Argon, water vapor was introduced into the reactor at a rate of 2.5 mL min⁻¹ using a Bronkhorst CEM Evaporator system (volume fraction φ=50%) for 1 hour. This step was followed by an irradiation step with a 200 W Xenon lamp under an Argon flow at a rate of 5 mL min⁻¹ for 2 hours. Hydrogen evolution was analyzed by a mass spectrometer (ThermoStar GSD 320 T1 from Pfeiffer Vacuum) and a gas chromatograph (Agilent 8890 GC system). The steps of water vapor introduction and light irradiation were repeated 2 additional times. In the 3 cycles performed, an average of 5.7 mL g⁻¹ H₂ were produced as shown in FIG. 9.

1. A catalytic process comprising the steps of:
i. providing a catalyst comprising atomic quantum clusters (AQCs) consisting of 3 to 10 metal atoms and a metal oxide, wherein the AQCs are deposited on the surface of the metal oxide; and;
ii. contacting said catalyst with a reducing agent at a temperature between room temperature and 800° C. , to release oxygen from the metal oxide;
iii. optionally contacting said catalyst with an oxidizing agent for at least partially replenishing the oxygen in the metal oxide;
wherein said reducing agent is selected from an inert atmosphere, a reductive atmosphere or a mixture thereof.

2. The process according to claim 1, wherein the AQCs consist of 5 metal atoms.

3. The process according to claim 1 or 2, wherein the metal of the AQCs is selected from Ag, Cu, Au, Pt, Pd, Fe, Co, Ni or their bi-metal and multi-metal combinations.

4. The process according to claim 3 wherein the metal of the AQCs is Cu, Ag, Pt or their bi-metal and multi-metal combinations.

5. The process according to claim 1 wherein the metal oxide is selected from the group consisting of TiO₂, WO₃, MnO₂, Mn₂O₃, Co₃O₄, Fe₂₀₃, Fe₃O₄, CoFe₂O₄, NiFe₂O₄, ZnFe₂O₄, FeAl₂O₄, BiFeO₃, FeAl₂O₄, CoAl₂O₄, La₂O₃, CeO₂, Ce₂O₃, Ce₃O₄, Nd₂O₃, Sm₂O₃, Eu₂O₃, Gd₂O₃, LaMnO₃, La₁₋ₓSrₓMnO₃ and La₁₋CeₓMnO₃.

6. The process according to claim 1, wherein the reducing agent is selected from the group consisting of vacuum; a partial pressure of oxygen of less than 1 bar; an inert gas; a reductive gas; or a combination thereof.

7. The process according to claim 6, wherein the reducing agent is an inert gas, a reductive gas or a combination thereof.

8. The process according to claim 1, wherein step (ii) is performed at room temperature.

9. The process according to claim 1, wherein step (ii) is performed at a temperature of 250 to 600° C. and the reducing agent is an inert gas selected from Ar, He or a mixture thereof.

10. The process according to claim 1, wherein step (ii) is performed at a temperature of 250 to 600° C. and the reducing agent is a reductive gas such as hydrogen, CH₄ or a mixture thereof.

11. The process according to claim 1, wherein step (ii) further comprises irradiating the catalyst with light.

12. The process according to claim 1, further comprising a step (iii) of contacting the catalyst with an oxidizing agent for at least partially replenishing the oxygen in the metal oxide, wherein the oxidizing agent is selected from air, carbon dioxide, water or a combination thereof.

13. The process according to claim 12, said process comprising:
i. providing a catalyst comprising atomic quantum clusters (AQCs) consisting of 3 to 10 metal atoms and a non-volatile metal oxide, wherein the AQCs are deposited on the surface of the non-volatile metal oxide;
ii. contacting said catalyst with an inert atmosphere at a temperature between room temperature and 800° C. to release oxygen from the metal oxide, while irradiating said catalyst with light; and
iii. contacting said catalyst with water substantially in the absence of light for at least partially replenishing the oxygen in the metal oxide and produce hydrogen.

14. The process according to claim 12, wherein step (iii) is performed at a temperature of 350 to 600° C.

15. An oxygen deficient catalyst obtainable by a process comprising the steps of:
i. providing a catalyst comprising atomic quantum clusters (AQCs) consisting of 3 to 10 metal atoms and a metal oxide, wherein the AQCs are deposited on the surface of the metal oxide; and
ii. contacting said catalyst with a reducing agent at a temperature between room temperature and 800° C. to release oxygen from the metal oxide;
wherein said reducing agent is selected from an inert atmosphere, a reductive atmosphere or a mixture thereof; and
wherein said catalyst has an excess of oxygen vacancies of at least 0.1% relative to the stoichiometric composition of the metal oxide.

16. The catalyst according to claim 15, wherein the metal AQCs are monodisperse AQCs.

17. (canceled)

18. The catalyst according to claim 15, wherein said catalyst comprises Cu or Ag AQCs consisting of 5 metal atoms deposited on the surface of CeO₂.

19. A catalytic composition comprising:
a) a catalyst comprising atomic quantum clusters (AQCs) consisting of 3 to 10 metal atoms and a metal oxide, wherein the AQCs are deposited on the surface of the metal oxide; and
b) a reducing agent selected from an inert atmosphere, a reductive atmosphere or a mixture thereof;
wherein the catalyst has an excess of oxygen vacancies of at least 0.1% relative to the stoichiometric composition of the metal oxide.

20. The catalytic composition according to claim 19, wherein the metal AQCs are monodisperse metal AQCs.

21. (canceled)

22. The catalytic composition according to claim 19, wherein the catalyst comprises Cu or Ag AQCs consisting of 5 metal atoms deposited on the surface of CeO₂.

23. The catalytic composition according to claim 19, wherein the reducing agent is an inert gas, preferably selected from argon, helium, nitrogen or a mixture thereof; and/or a reductive gas selected from H₂, CO, methane or a mixture thereof.

24. A method for performing chemical looping reactions comprising the step of depositing a catalyst comprising AQCs consisting of 3 to 10 metal atoms on the surface of a the metal oxide as oxygen carriers

25. The method according to claim 24, wherein the chemical-looping reaction is selected from the group consisting of chemical looping combustion, syngas production, water-gas shift reaction, steam methane reforming, selective oxidation of hydrocarbons, hydrogen generation, photo/thermochemical water-splitting, thermochemical water-splitting and thermochemical carbon dioxide splitting.
